(19) United States
(12) Patent Application Publication
(10) Pub. No.: US 2025/0366802 A1
(43) Pub. Date: Dec. 4, 2025

(54) DEVICE FOR SUPPORTING AN INFANT FOR MEDICAL PROCEDURES AND METHOD OF USE

(71) Applicants: Yoseph Adie, Whittier, CA (US); Marilu Venegas, Whittier, CA (US); Mohammad Amin Adie, Whittier, CA (US)

(72) Inventors: Yoseph Adie, Whittier, CA (US); Marilu Venegas, Whittier, CA (US); Mohammad Amin Adie, Whittier, CA (US)

(21) Appl. No.: 19/228,378

(22) Filed: Jun. 4, 2025

Related U.S. Application Data
(60) Provisional application No. 63/656,023, filed on Jun. 4, 2024.

(51) Int. Cl. A61B 6/04; A61B 6/10

(52) U.S. Cl. CPC A61B 6/0421; A61B 6/10; A61B 2503/045; A61B 2560/04

(57) ABSTRACT

The present invention relates to a device designed to safely and securely hold newborn infants during radiographic imaging and medical procedures. This device aims to immobilize neonates to prevent motion artifact during imaging while also protecting healthcare professionals from unnecessary radiation exposure, offering a safer alternative for conducting radiographic imaging on newborns and pediatric population. The device may be constructed primarily of fiberglass, or any medically graded radiolucent materials providing a lightweight, durable, and radiolucent structure. It can be padded with a soft cushion and incorporates a series of adjustable constraints to comfortably and securely hold the baby in place, allowing for precise positioning during radiographic imaging and medical procedures.

The present invention claims priority as a non-provisional of U.S. provisional application 63/656,023 filed on Jun. 4, 2024, presently pending. The contents of the application are hereby incorporated by reference.

### TECHNICAL FIELD OF THE INVENTION

The present invention relates to patient securing devices, and more specifically to a patient securing device for medical procedures.

### BACKGROUND

The pediatric population, especially premature infants, are at high risk of respiratory distress and gastrointestinal comorbidities. They often require frequent imaging to assess their lungs, abdomen and sometimes bones. Currently, there are no suitable devices in the market to immobilize premature neonates during radiographic imaging. In order to prevent motion artifacts during imaging, a healthcare professional is required to hold the baby's arms and legs to stabilize. Inevitably, this exposes the health care professional to radiation leading to an increased risk of cancer and malformation of the fetus if the healthcare professional is pregnant.

Presently, devices for immobilization of adults during transportation after trauma and for infants/children while sitting for imaging exist. However, there is no suitable device available for premature and term neonates.

### SUMMARY

A device for supporting an infant for medical imaging and procedures is provided, which includes at least one support board with a plurality of slots arranged in the support board to pass adjustable straps through. The device may also include a head rest arranged on the support board for holding the infant's head.

A method for supporting an infant for medical imaging or procedures is also provided, including placing the infant on a support board, situating the infant's head on a head rest on the support board and securing the infant to the support board with adjustable straps passing through a plurality of slots arranged on the support board.

### BRIEF DESCRIPTION OF THE DRAWINGS

The invention, together with the above and other objects and advantages, will be best understood from the following detailed description of the preferred embodiment of the invention shown in the accompanying drawings, wherein:

FIG. 1 is a top view of an embodiment of the securing device, for supporting an infant on its back.

FIG. 2A is a front perspective view of an alternate embodiment of the securing device, for supporting an infant on its side.

FIG. 2B is an illustration of an infant secured in the device shown in FIG. 2A.

FIG. 3 is an image of an infant secured in the embodiment shown in FIG. 1.

FIG. 4A is a front perspective image of an infant secured in the embodiment shown in FIG. 2A.

FIG. 4B is a left side perspective image of an infant secured in the embodiment shown in FIG. 2A.

FIG. 5 is a front perspective image of the device shown in FIG. 2A.

FIG. 6 is a bottom perspective image of the device shown in FIG. 2A.

FIG. 7 is a rear perspective image of the device shown in FIG. 2A.

### DETAILED DESCRIPTION

The device is a fixture for securely holding infants and newborns during radiography imaging or other medical procedures consisting of a base structure and adjustable constraints. The structure can be made of but is not limited to carbon fiber composites ensuring a lightweight, durable, and radiolucent design. It is equipped with a series of adjustable constraints that can be customized to securely hold the baby in place during imaging to prevent motion artifacts and potential radiation exposure to healthcare professionals.

The device features a padded platform for the baby to lie on, ensuring comfort and safety. The platform is adjustable to accommodate babies of different sizes. Additionally, the device includes a headrest to further secure the baby's position during the procedure. The fixture includes an embodiment for securing while the baby is laying on its side and another embodiment for when the baby is laying supine.

Each platform provides restraints for immobilization of the infant, based on the laying profile type. The whole structure is studded with slots strategically placed to allow for restraints to be threaded through, providing proper and comfortable securing of the infant.

The adjustable constraints are designed to gently but securely hold the baby's arms, legs, head, and torso in place. They can be easily adjusted to ensure a snug fit without causing discomfort to the baby. The device can be easily slid under the patient and allows for minimal handling of micro preemie newborns, reducing the risk of intraventricular hemorrhage (IVH). Overall, the structure of the device can be manufactured through but is not limited to machining or molding.

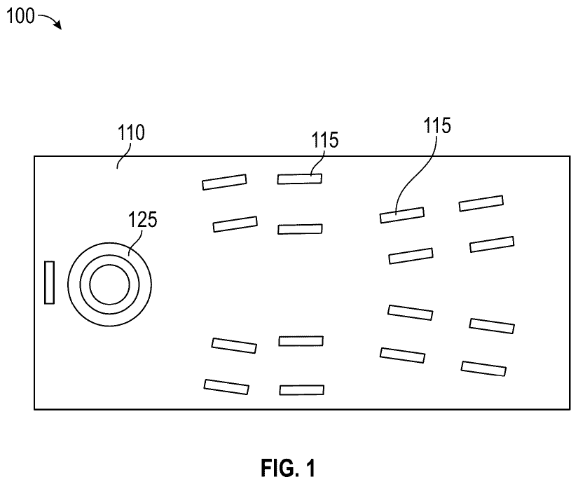

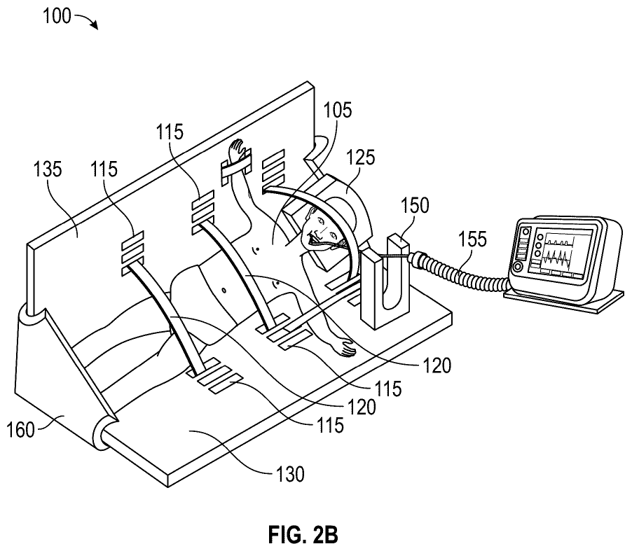

Referring to FIGS. 1 and 3, an embodiment of the device 100 for supporting an infant on the infant's back is illustrated. The device includes a base support board 110 with slots 115 arranged to pass adjustable straps (not shown) through the base support board 110. The device also includes a head rest 125 attached to the base support board 110 for supporting the infant's head. The base support board 110 is preferably constructed of fiberglass or any type of medically graded radiolucent material that allows transparency for medical imaging. The head rest 125 is preferably U-shaped for easy placement and comfort of the infant 105 but may be other shapes such as a donut-shape as a non-limiting example. The head rest 125 can also be provided in different sizes.

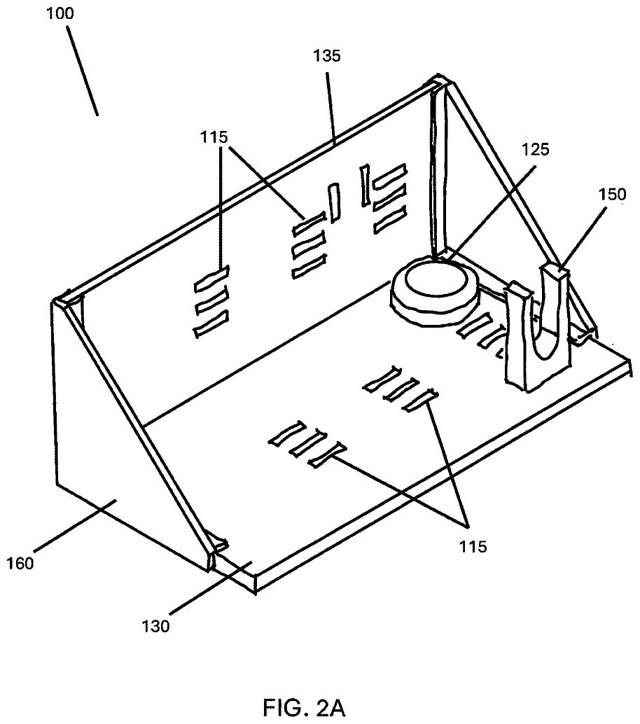

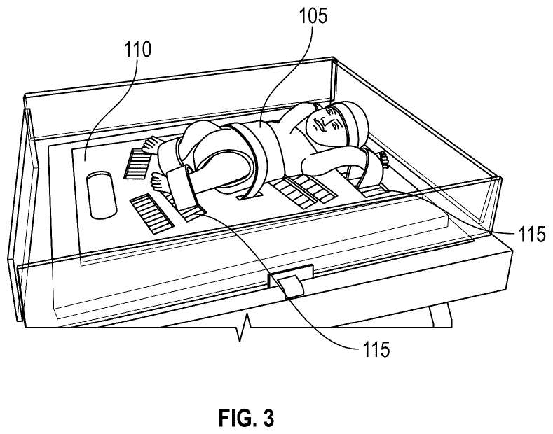

Referring to FIGS. 2A, 2B, 4A and 4B, an alternate embodiment of the device 100 is illustrated, for placement of the infant 105 on the infant's side. In this embodiment, a first support board 130 has slots 115 arranged to pass adjustable straps 120 through the first support board 130 in an arrangement for securing the infant 105 comfortably on the infant's side. A second support board 135 is arranged approximately perpendicular, also referred to as substantially perpendicular, and adjacent to the first support board 130, to create a safe combined L-shaped surface to lay and secure the infant. The second support board 135 also has slots 115 strategically placed to pass the adjustable straps 120 for securing the infant 105. The first support board 130 has the attached head rest 125 to hold and stabilize the infant's head. The second support board 135 may also have a head rest for further support of the infant's head. An extension 150 may be attached to the first support board 130, to hold an endotracheal tube (ETT) 155 and prevent the ETT from kinking, and for easy viewing of the ETT. Alternatively, the extension 150 may be attached to the second support board 135.

Still referring to FIGS. 2A, 2B, 4A and 4B, the first support board 130 is connected to the second support board 135 with one or more braces 160 for fixing the first support board against the second support board and for allowing the second support board to easily fold against the first support board for storage or transport. Alternatively the braces 160 can be fused to the first support board 130 and the second support board 135. The brace 160 may be a joining piece with perpendicular slots 165 to fit the first and second support boards 130, 135.

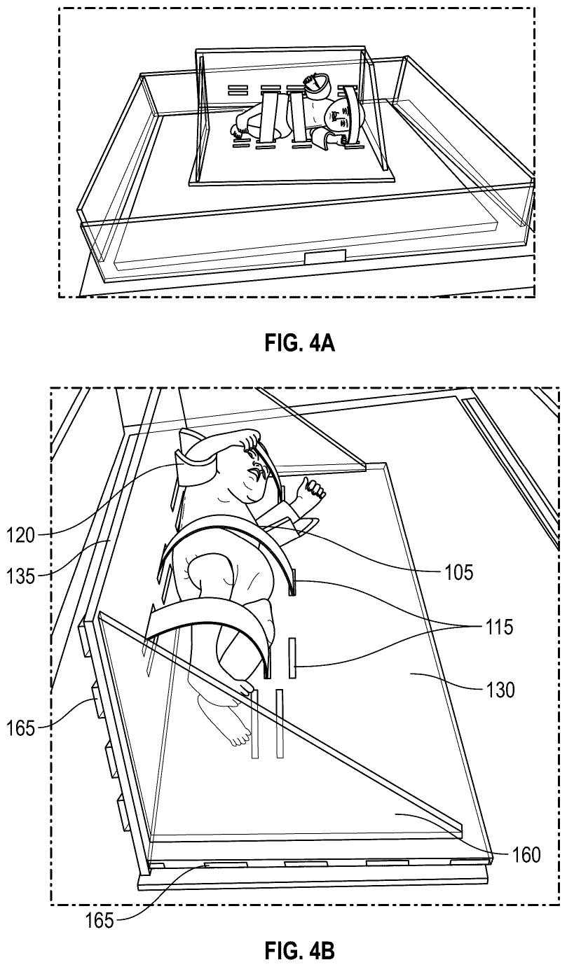

Referring to FIGS. 5, 6 and 7, frame 170 may be attached to the first support board 130 to raise the first support board by approximately 1 inch. Slots 175 in the frame 170 aligned with the slots 115 in the first support board 130 enable passing the straps 120 through.

The head rest 125 is preferably U-shaped for the comfort of the infant and to readily accommodate the head and neck of the infant. Alternatively, the head rest 125 may be donut shaped or another shape to optimize the comfort and security of the infant.

The support boards, 110, 130, 135 are preferably constructed of a material transparent to medical imaging. For example, fiberglass is a non-limiting example of a material for construction.

Dimensionally, the support boards 110 130, 135 may each be but are not limited to approximately 30-40 cm wide by 60-75 cm long (approximations herein are +/−10%). The first support board 130 is attached to the second support board 135 using snap or other form of supports to allow for proper stowaway and support or the first and second support boards can be fused. The first and second support boards 130, 135 may be approximately 1 cm thick. The compact structure enables it to be positioned through the sides of the incubator, allowing the top of the incubator to remain closed during placement of the device, allowing temperature stability as well as providing minimal stressful stimulation to newborns born under 30 weeks. The device can also be used in the radiology department, neonatal, pediatric or cardiac intensive care units.

A method for using the device 100 is also provided herein. An infant requiring medical care is placed on the infant's back on the base support board 110 with the infant's head resting on the head rest 125 as illustrated in FIG. 1. The infant is secured using adjustable straps passed through slots 115 in the base support board.

In an alternative embodiment of the method, as illustrated in FIGS. 2A and 2B, the infant 105 is placed on its side on a first support board 130. A second support board 135 that is perpendicular to the first support board supports the back of the infant 105. A head rest 125 on the first support board 130 is used to help hold the infant's head in place. The head rest 125 is preferably U-shaped for the comfort of the infant 105 or may be other shapes such as donut-shaped. The infant 105 is secured to the first and second support boards 130, 135 using adjustable straps 120 passed through slots 115 strategically placed in the support boards.

Referring to FIG. 3, the embodiment of the device shown in FIG. 1 is shown with an infant secured to the device.

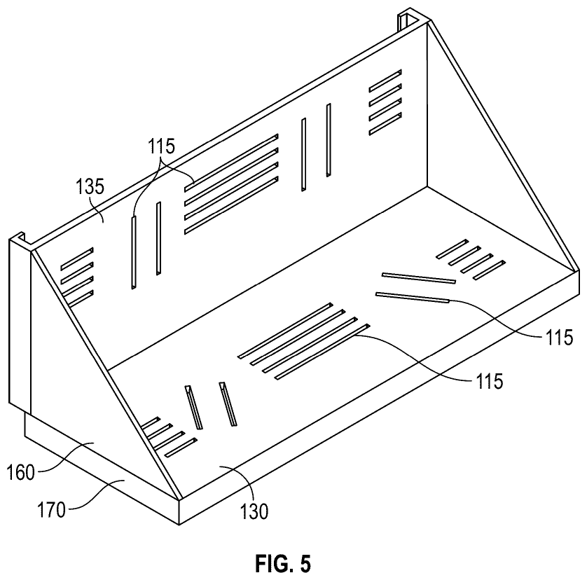

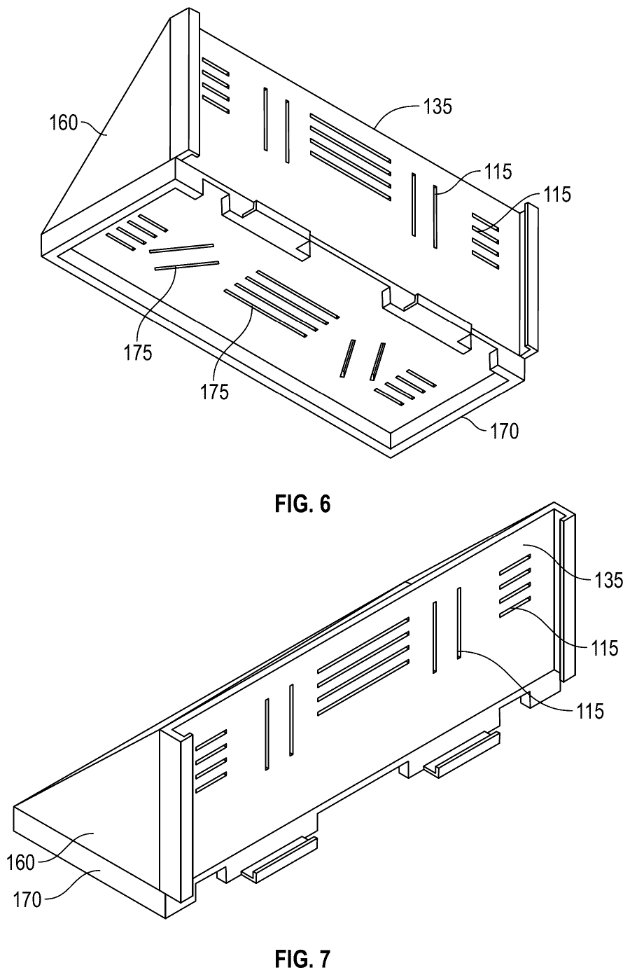

FIG. 4A and FIG. 4B are a front perspective image and a left side perspective image of an infant secured in the embodiment shown in FIG. 2A respectively. FIG. 5 is a front perspective image of the device shown in FIG. 2A. FIG. 6 is a bottom perspective image of the device shown in FIG. 2A and FIG. 7 is a rear perspective image of the device shown in FIG. 2A.

To store or transport the device illustrated in FIGS. 2A and 2B, the second support board 135 can be separated from or folded along the first support board 130 via a hinge or brace 160 attaching the first and second support boards, to provide a flat compact structure.

The present invention is useful to immobilize neonates during radiological imaging, medical procedures and treatment, for conditions affecting head, neck, spine, lungs, heart, abdomen, urogenital, bones and extremities. The device also eliminates unnecessary exposure of medical personnel to radiation.

What is claimed is:

1. A device for supporting an infant for medical procedures, the device comprising:
at least one support board having a plurality of slots arranged to pass adjustable straps through the at least one support board; and
a head rest arranged on the at least one support board for holding an infant head.

2. The device of claim 1, wherein the support board comprises a radiolucent material.

3. The device of claim 1, wherein the at least one support board comprises one support board and the head rest comprises a U-shaped head rest for supporting the infant head.

4. The device of claim 1, wherein the at least one support board comprises:
a first support board; and
a second support board arranged substantially perpendicular and adjacent to the first support board.

5. The device of claim 4, wherein the first support board is connected to the second support board with at least one brace for fixing the first support board against the second support board.

6. The device of claim 5, wherein the at least one brace comprises a joining piece with perpendicular slots to accommodate the first support board and the second support board.

7. The device of claim 4, further wherein the head rest comprises a donut-shaped head rest on the first support board and a U-shaped head rest on the second support board.

8. A method for supporting an infant for medical imaging, the method comprising:
placing the infant on a support board;
situating an infant head on a head rest on the support board; and
securing the infant to the support board with adjustable straps passing through a plurality of slots arranged on the support board.

9. The method of claim 8, wherein the support board is manufactured of a radiolucent material, to provide for a medical imaging.

10. The method of claim 8, wherein placing the infant comprises placing the infant on the support board and situating the infant head comprises situating the infant head on a U-shaped head rest on the support board.

11. The method of claim 8, wherein placing the infant comprises placing an infant side on a first support board and the infant back against a second support board arranged substantially perpendicular and adjacent to the first support board.

12. The method of claim 11, further wherein the first support board is connected by a brace to the second support board and the first support board is folded against the second support board for storage.

13. The method of claim 12, wherein the brace is a joining piece with perpendicular slots for accommodating the first support board and the second support board.

14. The method of claim 11, further wherein placing the infant head comprises placing the infant head on a donut-shaped head rest on the first support board and against a U-shaped head rest on the second support board.
